(19) United States
(12) Patent Application Publication
(10) Pub. No.: US 2014/0127157 A1
(43) Pub. Date: May 8, 2014

(54) ANTIVIRAL ACTIVITY OF TYROSINE KINASE INHIBITORS AGAINST HEPATITUS C VIRUS

(71) Applicants: Erica Canino, Alameda, CA (US); Adam Feire, Madison, WI (US); Christopher Jones, Basel (CH); Paul W. Manley, Basel (CH)

(72) Inventors: Erica Canino, Alameda, CA (US); Adam Feire, Madison, WI (US); Christopher Jones, Basel (CH); Paul W. Manley, Basel (CH)

(73) Assignee: Novartis AG, Basel (CH)

(21) Appl. No.: 14/071,451

(22) Filed: Nov. 4, 2013

Related U.S. Application Data
(60) Provisional application No. 61/722,381, filed on Nov. 5, 2012.

(51) Int. Cl. A61K 31/506; A61K 31/517; A61K 31/5377; A61K 38/21; A61K 31/496; A61K 31/404; A61K 31/7056; A61K 31/5025; A61K 31/4412

(52) U.S. Cl. CPC A61K 31/506; A61K 31/5025; A61K 31/517; A61K 31/5377; A61K 31/4412; A61K 31/496; A61K 31/404; A61K 31/7056; A61K 38/21
USPC 424/85.4; 514/275; 514/255.05; 514/248; 514/266.4; 514/234.5; 514/346; 514/253.1; 514/414; 514/43

(57) ABSTRACT

Antiviral activity of Nilotinib against Hepatitis C virus.

### FIELD OF THE INVENTION

Compositions of and methods for treating and preventing hepatitis C virus infection.

### BACKGROUND OF THE INVENTION

Hepatitis C virus (HCV) is a blood-borne pathogen affecting nearly 3% of the world's population and is a leading indicator of liver transplantation. Chronic HCV infection, which occurs in approximately 60-80% of infected individuals, can progress to serious liver disease including cirrhosis and hepatocellular carcinoma. Current therapy consisting of a combination of pegylated interferon and ribavirin (PEG-Rib) is often poorly tolerated and has limited efficacy. Recently FDA-approved direct-acting antiviral (DAAs) agents are associated with higher cure rates but still depend on co-administration with PEG-Rib, and are therefore associated with significant side effects. As a result, efforts have been made to understand the complete HCV life cycle with the goal of identifying additional novel therapeutic targets. Kinases are a large family of proteins that catalyze the addition of phosphate groups to a variety of substrates, including protein and lipids. Kinases are involved in key regulatory steps in cellular biology, and are therefore not surprisingly a common target of anti-cancer therapeutics. Kinases also play important roles in the life cycles of many viruses, including HCV. Phosphorylation of the HCV protein NS5A by cellular kinases is thought to regulate RNA replication and virus assembly. In addition, the cellular lipid kinase phosphatidylinositol 4-kinase alpha has been shown to be essential for HCV replication. Recently, epidermal growth factor receptor (EGFR) and other kinases have been implicated in virus entry. Indeed, small molecule inhibitors of EGFR, including Erlotinib and Gefitinib, inhibit HCV entry in vitro. Nilotinib is a tyrosine kinase inhibitor having selectivity towards the tyrosine kinase activity of the Abelson, ABL1 and Abelson-related, ABL2 tyrosine kinases, the mixed-lineage ZAK kinase, as well as the tyrosine kinase activity of the discoidin domain (DDR), stem cell factor (KIT), ephrin (EPH) and platelet-derived growth factor (PDGFR) receptor kinases, but not the epidermal growth factor receptor kinase, EGFR. Here we demonstrate that tyrosine kinases, including nilotinib, potently inhibit HCV, through a mechanism likely involving virus entry.

### SUMMARY OF THE INVENTION

An embodiment of the invention provides a method of treating hepatitis virus C in an infected patient or prophylactically treating hepatitis virus C in a patient comprising providing an effective amount of a tyrosine kinase inhibitor to a patient.

Another embodiment of the invention provides a method wherein the tyrosine kinase inhibitor is selected from nilotinib, radotinib, ponatinib, erlotinib, gefitinib, regorafenib, erlotinib, sorafenib, masitinib and sunitinib and the pharmaceutically acceptable salts thereof.

Another embodiment of the invention provides a method wherein the tyrosine kinase inhibitor is a c-Abl tyrosine kinase antagonist preferably nilotinib.

Another embodiment includes methods of treating hepatitis virus C in an infected patient or prophylactically treating hepatitis virus C in a patient comprising providing an effective amount of a tyrosine kinase inhibitor to a patient together with an additional active agent. Suitable additional active agents include direct acting, such as viral protease, polymerase and NS5A inhibitors, and/or host targeting antiviral agents which include but are not limited to cyclophilin inhibitors, ribavirin and interferons which include interferon-alpha-2a and 2b and type III interferons, such as interferon lambda.

The tyrosine kinase inhibitor may be provided by any pharmaceutically acceptable method, such as administration as an oral dosage form or intravenously. In certain embodiments the tyrosine kinase inhibitor is provided to an infected patient together with supportive treatment such as intravenous fluids given to prevent or reverse dehydration and/or transfusions of platelets.

### BRIEF DESCRIPTION OF THE DRAWING

FIG. 1. The effect of agents added before or after entry of HCV into cells. The presence of compound during both binding and post-binding are indicated by black bars; white bars indicate compounds present only at post-binding. DMSO, dimethyl sulfoxide; 5Ainh, HCV NS5A inhibitor (50×EC50); nilotinib (50×EC50); α-CD81, anti-CD81 antibody (1 μg/mL); α-IgG, iso-type control antibody (1 μg/mL).

FIG. 2. The effect of agents added during entry of HCV into cells-. HCV kinetic entry assay in the presence of compounds. 5Ainh, black circle; heparin, black diamond; α-CD81, white triangle; ConA, black square; nilotinib, white circle; Baf, black triangle; Gef, white square.

### DETAILED DESCRIPTION OF THE INVENTION

Compounds are described using standard nomenclature. Unless defined otherwise, all technical and scientific terms used herein have the same meaning as is commonly understood by one of skill in the art to which this disclosure belongs.

The use of the terms “a”, “an”, and “the” and similar referents in the context of the disclosure (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the disclosure unless otherwise claimed. “About” indicates an approximate amount, including the quantity it modifies. No language in the specification should be construed as indicating any non-claimed element as essential to the practice of the invention.

An “active agent” is any compound, element, or mixture, that when administered to a patient alone or in combination with one or more other agents confers a therapeutic benefit on the patient. When the active agent is a compound, solvates (including hydrates) of the free compound or salt, crystalline and non-crystalline forms, as well as various polymorphs or the compound are included. For example, an active agent can include optical isomers of the compound and pharmaceutically acceptable salts thereof either alone or in combination.

The term “dosage form” denotes a form of a pharmaceutical composition that contains an amount sufficient to achieve a therapeutic effect with a single administration. The term “oral dosage form” is meant to include a unit dosage form prescribed or intended for oral administration. An oral dosage form may or may not comprise a plurality of subunits such as, for example, microcapsules or microtablets, packaged for administration in a single dose.

The term “effective amount” means an amount effective, when administered to a human or non-human patient, to provide any therapeutic benefit such as an amelioration of symptoms, e.g., an amount effective to decrease the symptoms of viral infection, and preferably an amount sufficient to decrease the symptoms of hepatitis C virus infection. An “effective amount” may also be an amount sufficient to decrease viral load or viral antibodies in the patient's blood or tissues. Viral load can be determined in patient blood using quantitative reverse transcriptase polymerase chain reaction (qRT-PCR).

A “patient” is any human or non-human animal in need of medical treatment. In preferred embodiments the patient is a human patient determined to have hepatitis C virus infection. Medical treatment can include treatment of an existing condition, such as a disease or disorder, or prophylactic or preventative treatment.

“Pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC-(CH₂)n-COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

Tyrosine kinase inhibitors include both biological molecules and small molecules. Types of tyrosine kinase inhibitors include ABL, EGFR and multi-targeted tyrosine kinase inhibitors that reduce the activity of ABL tyrosine kinase whether by reducing the levels of ABL protein as in the case of ABL tyrosine kinase specific siRNAs or by interacting directly with the ABL tyrosine kinase protein and thereby reducing its enzymatic activity (allosteric, non-ATP competitive ABL inhibitors).

An effective ABL tyrosine kinase antagonists for use in this method includes nilotinib (CAS Reg. No. 641571-10-0). The structure of nilotinib is as follows:

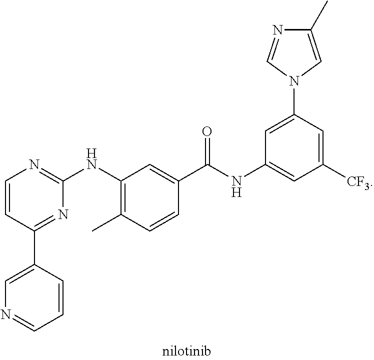 [SMILES: Cc1cn(-c2cc(NC(=O)c3ccc(C)c(Nc4nccc(-c5cccnc5)n4)c3)cc(C(F)(F)F)c2)cn1]

Nilotinib is described in U.S. Pat. No. 7,169,791 and is hereby incorporated by reference at columns 14-17 and 26-52 for its teachings regards nilotinib and its analogues.

Other tyrosine kinase inhibitors useful in the methods of treatment described herein include dasatinib (CAS Reg. No. 302962-49-8), bosutinib (CAS Reg. No. 380843-75-4), radotinib (CAS Reg. No. 926037-48-1), ponatinib (CAS Reg. No 943319-70-8), erlotinib (CAS Reg. No. 183321-74-6), gefitinib (CAS Reg. No. 184475-35-2), regorafenib (CAS Reg. No. 755037-03-7) sorafenib (CAS Reg. No. 284461-73-0), masitinib (CAS Reg. No. 790299-79-5) and sunitinib (CAS Reg. No. 557795-19-4) and their pharmaceutically acceptable salts thereof.

The infectious cell culture system for HCV (HCVcc) is based on a unique full length strain of genotype 2a derived from a Japanese patient with fulminant hepatitis (JFH-1). HCVcc allows the complete virus life cycle to be studied in cell culture. A chimeric strain of JFH-1, termed Jc1JFH, is capable of more robust growth in tissue culture and can be engineered to encode a reporter gene. The activity of compounds such as nilotinib against HCVcc can be investigated using a Jc1JFH reporter virus encoding luciferase (Table 1). Naïve Huh-7.5 cells are treated with serially diluted compounds in DMSO for 2 h prior to infection with HCVcc. At 72 h post infection, luciferase activity of infected cell lysates is measured and 50% effective concentration (EC₅₀) values are determined for each compound. The concentration that resulted in 50% cellular cytotoxicity (CC₅₀) is determined by CellTiter Glo assay (Promega) using uninfected, compound-treated cells. Both EC₅₀ and CC₅₀ values are normalized to cells treated with DMSO alone. Nilotinib shows EC₅₀ and CC₅₀ values nilotinib of 0.04 and 8.8 μM, respectively. In comparison, imatinib shows activity with an EC₅₀ value of 4.6 μM against HCVcc (Table 1). Together this data indicates that nilotinib is a potent inhibitor of HCV.

To explore the mechanism behind the anti-viral activity of nilotinib, the HCV replicon (HCVrep) system can be employed. HCVrep comprises a stable cell line harboring a sub-genomic replicon encoding a selectable marker (e.g., neomycin phosphotransferase), a reporter gene (e.g., luciferase) and the minimal HCV replicase proteins, NS3 to NS5B. Since the subgenomic replicon is non-infectious, the HCVrep system is used to study viral RNA replication independently of other stages of the life cycle, including virus entry/assembly/release. Thus, compounds that are active against both HCVcc and HCVrep are likely inhibitors of HCV RNA replication, while compounds that inhibit HCVcc but not HCVrep likely target other aspects of the virus life cycle (e.g., entry). To determine the effects of compounds such as nilotinib on HCV RNA replication, a stable JFH-1-based replicon cell line encoding luciferase was established. The HCVrep cell line is treated with compounds for 72 h and EC₅₀ and CC₅₀ values are determined in parallel, as above. Nilotinib is 100-fold more potent against HCVcc than HCVrep; imatinib did not show activity at the concentrations tested against the replicon (Table 1). The relatively low potency of nilotinib against HCVrep relative to infectious virus suggests that the inhibition of HCV activity is through a mechanism of action (MOA) that does not involve viral RNA replication.

TABLE 1
Activity of nilotinib against HCVcc and HCVrep

| | HCVcc | HCVcc | HCVrep | HCVrep |
|---|---|---|---|---|
| Compound | (EC₅₀, μM) | (CC₅₀, μM) | (EC₅₀, μM) | (CC₅₀, μM) |
| Nilotinib | 0.04 | 8.8 | 4.2 | 8.2 |
| Imatinib | 4.6 | 26 | 20 | 24 |

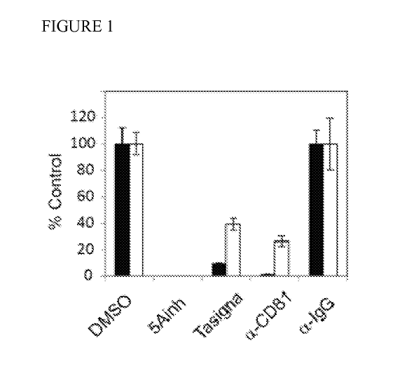

To further explore the MOA of compounds such as nilotinib, a time of addition experiment is performed to determine whether the compound acted at an early (i.e., entry) or late (i.e., assembly/release) stage of the virus life cycle. Naive Huh7.5 cells are infected for 4 h with HCVcc in either the presence (treatment 1) or absence (treatment 2) of compound. At 4 h post-infection, compound is then added to the treatment 2 cells. At 72 h post infection, luciferase activity is determined. An inhibitor of viral entry (antibody targeting CD81) and an HCV replication inhibitor (5Ainh) are used as comparators. The results of the time of addition experiment are shown in FIG. 1. The HCV replication inhibitor is equally potent when added during or after HCV virus entry. In contrast, nilotinib is significantly more potent when added during the first 4 h of infection (treatment 1) relative to addition post-virus entry (treatment 2). Similar behavior is seen with the control anti-CD81 antibody, suggesting that nilotinib targets an early step in the HCV life cycle (i.e., virus entry).

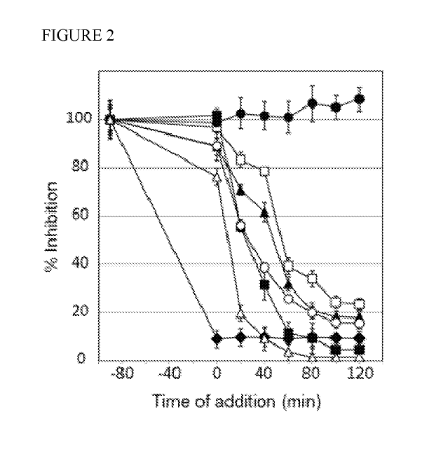

The HCV entry pathway is complex and involves multiple host factors. Although the exact order of events is not completely understood, HCV entry can be separated into three temporally distinct stages; virus binding, early post-virus binding, and late post-virus binding. Virus binding occurs through non-specific cell surface interactions that can be blocked by heparin sulfate. Early post-virus binding involves particle interaction with multiple entry factors including SRB1, CD81 and at least two tight junction proteins. Late post-virus binding events culminate in particle-endosomal fusion and require endosomal acidification as well as additional host factors, including EGFR. To determine when in the viral entry pathway compounds such as nilotinib may be acting, a kinetic entry assay can be performed. In this assay, infection is synchronized by incubation of cells and virus together for 1.5 h at 4° C. to allow binding, but not subsequent entry steps. After synchronization, unbound virus is removed by washing, and cells are shifted to 37° C. to initiate post-binding events. Compounds are added either immediately prior to (time zero), or at 20 min intervals post-temperature shift for up to 2 h. Compounds are removed at 4 h post-addition and luciferase activity is measured after 48 h of infection. Controls are included, which are known to block various steps of the HCV entry process: heparin sulfate (virus binding), anti-CD81 antibody (early post-virus binding), concanamycin A (ConA; late post-virus binding), bafilomycin (Baf; late post-virus binding), gefitinib (Gef; late post-virus binding). Percent inhibition is determined relative to treatment with compounds during synchronized virus binding at 4° C. Representative data of percent inhibition versus time of addition (FIG. 2) is used to estimate half-maximal times (t_{1/2}) to inhibit HCV entry. Heparin effectively blocks infection only when present during virus binding, while longer t_{1/2} values are seen for the inhibitors targeting later entry steps. Nilotinib shows similar inhibition kinetics to that of ConA and Baf, which are both inhibitors of endosomal acidification, indicating that nilotinib effects virus fusion. Gef, a small molecule inhibitor of EGFR, inhibited HCV entry at a later time point than nilotinib.

A summary of other tyrosine kinase inhibitors is provided in Table 2.

| Compound Name | EC50 (uM) | CC50 (uM) | CC50/EC50 |
|---|---|---|---|
| Nilotinib | 0.04 | 8.77 | 219.25 |
| Rodotinib | 0.04 | >2.5 | n/c |
| Ponatinib | 0.05 | 0.58 | 11.6 |
| Gefitinib | 0.08 | 10.6 | 132.5 |
| Regorafenib | 0.33 | >2.5 | n/c |
| Erlotinib | 0.47 | >25 | n/c |
| Sorafenib | 0.47 | 5.75 | 12.23 |
| Masitinib | 0.77 | 6.27 | 14.58 |
| Sunitinib | 0.94 | 3.54 | 3.77 |
| Non-ATP competitive ABL | 1.6 | >25 | n/c |
| inhibitor | | | |
| Dasatinib | 1.48 | 18.72 | 12.65 |
| Imatinib | 4.63 | 26.36 | 5.69 |

It has been discovered that nilotinib, an inhibitor of the cellular kinase ABL that is approved to treat Philadelphia chromosome positive chronic myeloid leukemia, is a potent inhibitor of HCV in cells.

Mechanistic analyses in cell culture indicate that nilotinib acts during viral entry, possibly at a late step involving virus-endosome fusion.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A method of treating hepatitis virus C in a patient or prophylactically treating hepatitis virus C in a patient comprising providing an effective amount of a tyrosine kinase inhibitor to a patient.

2. The method according to claim 1, wherein the tyrosine kinase inhibitor is a tyrosine kinase antagonist.

3. The method according to claim 1, wherein the tyrosine kinase inhibitor is selected from nilotinib, radotinib, ponatinib, erlotinib, gefitinib, regorafenib, erlotinib, sorafenib, masitinib and sunitinib and the pharmaceutically acceptable salts thereof.

4. The method according to claim 1, wherein the tyrosine kinase inhibitor is a c-Abl tyrosine kinase antagonist.

5. The method according to claim 4, wherein the c-Abl tyrosine kinase antagonist is nilotinib and the pharmaceutically acceptable salts thereof.

6. The method of any one claims 1, wherein the tyrosine kinase inhibitor is provided together with an additional active agent.

7. The method according to claim 6 wherein said additional active agent is ribavirin and/or interferon.

8. The method of any one of claims 1, wherein the tyrosine kinase inhibitor is provided to a patient as oral dosage form.

9. The method of any one of claim 1, wherein the tyrosine kinase inhibitor is provided to a patient by intravenous administration.

10. The method of any one of claim 1, wherein the tyrosine kinase inhibitor is provided together with intravenous and/or transfusions of platelets.
